(19) United States
(12) Patent Application Publication
(10) Pub. No.: US 2010/0129425 A1
(43) Pub. Date: May 27, 2010

(54) VAGINAL DELIVERY SYSTEM FOR MIRTAZAPINE

(75) Inventors: Wouter De Graaff, Oss (NL); Armin Szegedi, New York, NY (US)

Correspondence Address: ORGANON USA, INC.;c/o MERCK, 2000 Galloping Hill Road, Mail Stop: K-6-1, 1990, Kenilworth, NJ, 07033, US

(73) Assignee: N.V. Organon

(21) Appl. No.: 12/516,030

(22) PCT Filed: Nov. 21, 2007
(86) PCT No.: PCT/EP2007/062654

§ 371 Date: Jan. 20, 2010

Related U.S. Application Data
(60) Provisional application No. 60/860,753, filed on Nov. 22, 2006.

(30) Foreign Application Priority Data
Nov. 22, 2006 (EP) 06124593.2

(51) Int. Cl. A61K 9/00; A61K 31/55

(52) U.S. Cl. 424/432; 514/214.02; 424/400

(57) ABSTRACT

This invention relates to an extended release formulation comprising solid mirtazapine, which formulation is a vaginal device having a skin and which device comprises an inner compartment made of a thermoplastic polymer, which polymer is containing mirtazapine. The polymer is preferably made of ethylene-vinyl acetate copolymer.

The present invention relates to an extended release formulation comprising solid mirtazapine.

Mirtazapine is a widely used drug with therapeutic use in psychiatry, mainly for the treatment of major depression. For such disorders chronic use is necessary for therapeutic results. With dosage regimes based on prescription of tablets which have to be taken daily, it is very common that tablets are forgotten and that compliance of the patient with the treatment is less than desired. There is therefore a strong need for a very patient friendly extended release formulation of mirtazapine. In general, there are many extended release formulations available and most of them are based on implantation or injection of the formulation. Alternatives are patches for transdermal delivery. In the context of psychiatric drug treatment it is highly unusual to contemplate a vaginal delivery system for extended release, although the intravaginal route of administration was mentioned before in a broad list of possibilities for administering mirtazapine (WO 02/064735). Descriptions of vaginal delivery devices in general are in US 2003/0153983, WO 02/076426, WO 03/055424, U.S. Pat. No. 5,558,877 and U.S. Pat. No. 4,016,251.

The intravaginal route of administration has found use for contraceptive regimes or hormone replacement therapies which are exclusively aimed at treatment of the female person. Vaginal delivery devices are in particular known in the field of gynaecology for the delivery of hydrophobic steroidal drugs for contraceptive uses, such as exemplified in U.S. Pat. No. 4,292,965, WO97/02015, WO2004/103336, U.S. Pat. No. 4,469,671 and EP 0 876 815. A contraceptive vaginal ring is marketed under the trademark Nuvaring® by Organon, the Netherlands. Such rings are designed for the purpose of administering high potency steroids, for which drug delivery rates in the order of 0.01 to 0.5 mg/day are usually sufficient to obtain beneficial therapeutic effects. However, for mirtazapine therapeutically effective amounts to be delivered locally is much higher and ranges in the order of 0.1 to 60 milligrams a day.

Nevertheless there may be great advantage in providing an extended release formulation for mirtazapine in the form of a vaginal delivery device. The drug is used for therapeutic indications which occur more frequently in women, so that an extended release formulation which can only be used for women is still an important contribution to the art. For example, another drug for the treatment of depression, fluoxetineHCl, has been proposed for use in an extended release formulation in the form of a vaginal delivery system (WO 03/055424). The described device includes, as drug containing compartment, one or more channels in the surface or a pocket molded in the ring or a hollow toroid polydimethylsiloxane tubing for use. WO 2005/004837 describes a device with an inner drug containing compartment (reservoir) containing dispersed active agent and a sheath discontinuously surrounding the inner compartment. WO0170154 discloses a siloxane elastomer vaginal ring device with a bore located in the ring comprising an oxybutynin composition, wherein the bore runs from the surface of the ring into the ring. For non-steroidal drugs the choice for polysiloxane polymers relates to their high drug solubility and the well known high permeability of polysiloxane polymers (A. D. Woolfson, R. K. Malcolm, R. J. Gallagher, Journal of Controlled Release 91 (2003) 465-476). In addition, the diffusion coefficient for the same type of molecules in polysiloxanes is typically 100 to 200 times higher than the diffusion coefficient found in polyvinyl acetate copolymers (poly-EVA) (Treatise on controlled drug delivery; fundamentals, optimization, applications, edited by A. Kydonieus, Marcel Dekker Inc. New York, 1992. Typical diffusion coefficient for steroids, pp. 66-67).

Unexpectedly, it has now been found that an extended release formulation in the form of a vaginal delivery system can be prepared for mirtazapine with superior drug delivery characteristics in terms of high-release rate of mirtazapine, low-burst release, substantially constant release rate, in combination with a high drug substance efficiency and a duration of use of from one week up to 1 month, and which has optimal mechanical properties, in particular flexibility in the delivery system by avoiding the use of polysiloxane as taught in the prior art. The device according to the present invention also provides for a better range of options for the amount of mirtazapine to be released daily from the device.

The present invention provides for a vaginal device comprising solid mirtazapine, a skin and an inner compartment, which inner compartment is made of a thermoplastic polymer, which polymer is containing mirtazapine. Preferably, the skin is a substantially continuous cover over the inner compartment. Good results can be obtained when the inner compartment contains 5-80 wt % of mirtazapine. Optionally, the inner compartment comprises a core, which does not contain solid mirtazapine. Preferably, the inner compartment, and/or the skin, and/or the core or all three of these are made of ethylene-vinyl acetate copolymer. In a more specific embodiment an ethylene-vinyl acetate copolymer having a vinyl acetate content in the range of 6 to 40% is used.

Advantageous characteristics of the invention are that the device can easily be manufactured using extrusion techniques and is flexible in view of the small cross-sectional diameter if manufactured in the form of a ring. In addition to that, the extended release formulation according to the invention has an intrinsically safe design against dose-dumping. By application of a core in the inner compartment, the system allows for an improved drug substance efficiency.

The presence of mirtazapine in solid form provides for a sufficient and continuous supply of mirtazapine during release and the solid form prevents crystallisation of the drug on the outside of the device during manufacturing.

### Clarification of Terminology.

With a vaginal device a drug delivery system for insertion into the vagina of a woman is meant. The system has preferably the form of a ring, such that the delivery system has an elongated shape of which the two ends are joined together. The ring may comprise one or more loops and those loops may have various shapes, such as oval, ellipsoidal, toroidal, triangular, square, hexagonal, octagonal, etc. Alternatively, the system according to the invention is helically-shaped, which means the shape of a fiber helix with more than one loop and two ends which are not joined together. Mirtazapine is a well-known active compound which can be used for treatment of depression, sleep disorders, menopausal complaints etc. In particular for the latter use the ring according to the present invention is suitable. Depression occurs more frequently in women, so that the invention is in particular of benefit for the treatment of depressive disorders with mirtazapine. Mirtazapine is also known to be of benefit for women suffering from excessive hot flush, which makes the device according to the present invention particularly suitable for this group of patients. The vaginal route of administration of an extended release formulation in the form of a device improves compliance with drug treatment in view of the ease with which the formulation can be applied and removed by the women in need of treatment. Mirtazapine is available and can be used in the ring according to the invention, preferably as a base. Anyway, it should be in a non-ionised (neutral, uncharged) form when dissolving in the thermoplastic polymer of the inner compartment of the device, with a solubility therein of more than 0.1 wt %. This makes salts of mirtazapine usually unsuitable for use in the formulation according to the invention. Mirtazapine may be used in the form of its S- or R-enantiomer. The solid form required to obtain the high load of 5 to 80 wt % of mirtazapine in the polymer is preferably crystalline mirtazapine. The crystals will effectively be dispersed within the polymer of the inner compartment. Another reason to require the presence of solid mirtazapine is to obtain the extended delivery of mirtazapine from within the inner compartment as will be explained in more detail herein below.

With continuous skin is meant that the skin is continuously surrounding the mirtazapine containing compartment and is devoid of expressly provided parts in the skin for release of the drug. Thus, direct contact between vaginal tissue and drug compartment is minimised in order to avoid local irritation. The skin in substantially continuous in the sense that only incidental apertures may be present for example, the ends of a helically shaped system or apertures due to shear during manufacturing or due to incomplete closure of ring ends, but such openings are not purposefully introduced into the skin in order to facilitate the passage of mirtazapine through the skin. It is not excluded that the skin material may comprise some dissolved mirtazapine.

An inner compartment of the device is the compartment which contains the mirtazapine to be delivered to the patient and is covered by the skin. Therefore, there is no direct contact between the vaginal tissue and the inner compartment. The skin is the barrier protecting the vaginal tissue from undesirable local effects from the concentrated drug in the inner compartment. The inner compartment is formed by a thermoplastic polymer.

A core is an inner structure within the inner compartment and serves to reduce the drug containing space in the inner compartment. The core does not contain solid mirtazapine. It is not excluded, though, that the core material may comprise some dissolved mirtazapine. When mirtazapine is loaded into the inner compartment during the production process some mirtazapine may enter into the core. The core can be made of any suitable material such as a metal, a polymer or the same material as the polymer used for the inner compartment. The core can also contribute to the strength or flexibility of the device and to increase drug substance efficiency. In another context the inner compartment is also referred to as an intermediate layer when a core is present in the device.

The present invention provides for delivery rates of mirtazapine in the range of 0.1 to 60 mg/day for a period of use of from one week up to 1 or 2 months.

The characteristic of the invention may be understood and influenced by the following explanation and use thereof: Fick's law of diffusion governs the release of compounds. Vaginal rings are cylindrical reservoir/membrane designs of which the release rate can be described by the equation below. Suitable rings can therefore be made by an appropriate choice of the parameters that affect the release rate.

The release rate of a cylindrical reservoir/membrane design is:

$$\frac{d M}{d t} = \frac{2 π {LD}_{p} {K}_{p / s} Δ C}{Ln ( {r}_{0} / {r}_{i} )}$$

L=the length of the cylinder
Dₚ=the diffusion co-efficient of the compound in a skin polymer
K_{p/s}=partition coefficient of the compound between the skin and inner compartment
ΔC=the difference in concentration of dissolved mirtazapine between the inner compartment near the skin and the sink
r₀=is the overall radius, i.e. the radius of the cylinder including the skin
rᵢ=is the radius of the inner compartment (i.e. r₂/r₁=1) or of the core plus inner compartment (i.e. r₁, core comprising ring)

The equation shows that zero order release is obtained when the term on the right-hand side of the equation is constant, i.e. not a function of time.

It is shown in FIGS. 2 and 3 that release rates of mirtazapine of 7.5 to 25 mg/day can be achieved with the devices according to the invention having a skin substantially continuously covering the inner compartment.

Apparently, the solubility of mirtazapine in ethylene-vinyl acetate (EVA) of the inner compartment is such that the ΔC for mirtazapine is high enough to provide for fast release kinetics. The limiting factor in maintaining a substantially constant ΔC in a quasi steady state with a high release rate of mirtazapine, i.e. maintaining a substantially constant drug delivery from the device in the presence of a relatively thin skin with low barrier properties, is the supply of dissolved mirtazapine to the interface between the inner compartment and the skin. The supply (or referred to as release rate) is the result of a complex mass transport process determined by factors including the dissolution rate of mirtazapine into the polymer, which in turn is determined by the solubility of mirtazapine in the polymer and the surface area of the drug exposed to the polymer. The latter is determined by particle size, shape and drug content. Also the diffusion rate of mirtazapine through the polymer is an important factor for the dissolution and release rate. It has been found that devices having about 40 to 80 wt % of mirtazapine in the inner compartment not only provide for fast release rates but, when compared with devices comprising 5 to about 40%, in addition to that, provide for significantly more linear or substantially constant release kinetics. It is believed that with drug contents in the polymer above 40 wt. % drug particles can be close to each other within the polymer of the inner compartment. The structure formed by the dispersed solid particles in the polymer depends on drug content and additionally on particle size and shape. During drug release, the properties of the inner compartment itself change in time by the slow dissolution of the drug particles, apparently facilitating drug dissolution and transport rate resulting substantially constant high release rates. Probably the formation of improved diffusion pathways in the polymer by the progressively dissolving particles leaving voids in the polymer and, the simultaneous flow of aqueous liquids through the skin into the inner compartment filling the voids with water are important factors in achieving substantially constant release at high levels of drug content.

In the delivery devices of the invention mirtazapine is present in all polymer layers. When a drug in the manufacturing process of the system is loaded into the inner compartment, the drug diffuses during the production process and/or during storage of the system to the other polymer layer(s) up to equilibrium concentration. In line with the concept of the core comprising ring, for a ring without core the lengthening of the diffusion distance should also be kept as small as possible and the active compound should also be present in the solid form in order to obtain essentially zero-order release kinetics. Lengthening of the diffusion distance in case of the ring without core can be kept relatively small by keeping the cross-sectional diameter of the inner compartment relatively small. Such a small diameter also results in a relatively small volume of the inner compartment and hence, the amount of active compound, which is required to sustain the release for the intended period of use, is loaded in high concentration in the inner compartment.

A high concentration of active compound in the inner compartment of a ring without core also could be achieved in a large diameter ring, but this would require the use of a large excess of active compound, i.e. much more than required to sustain the release over the intended period of use and hence, this results in an economically and environmentally less attractive dose form with a low drug substance efficiency.

In analogy with a small inner compartment volume of the ring without core, a small inner compartment volume of the core comprising ring serves the purpose of concentrating the active compound in a relatively small polymer volume during processing.

The vaginal delivery system according to the present invention can provide a release rate of mirtazapine in the range of 0.1 to 60 mg/day for a period of use of from one week up to 1 month. Preferably the rate is in the range of 2 to 20 mg/day.

The thermoplastic polymer that can be used in making the drug delivery system according to the present invention may in principle be any extrudable thermoplastic polymer material suitable for pharmaceutical use, such as ethylene-vinyl acetate (EVA) copolymers, low density polyethylene, polyurethanes, and styrene-butadiene copolymers. In a preferred embodiment, ethylene-vinyl acetate copolymer is used due to its excellent mechanical and physical properties. The EVA copolymer may be used for the core, the intermediate compartment (inner compartment) as well as the skin and can be any commercially available ethylene-vinyl acetate copolymer, such as the products available under the trade names: Elvax, Evatane, Lupolen, Movriton, Ultrathene, Ateva, and Vestypar. These ethylene-vinyl acetate copolymers are available in different grades with respect to the amount of vinyl acetate present in the copolymer, for example, EVA 28 is a copolymer having a vinyl acetate content of 28%.

In one embodiment, at least the skin is made of ethylene-vinyl acetate copolymer. In a further embodiment, the core, the inner compartment, and the skin or the inner compartment and the skin (in a ring without core) are made of ethylene-vinyl acetate copolymers, which copolymers can each be of the same or different grades.

In another embodiment, the inner compartments are made of the same grade of ethylene-vinyl acetate copolymer. However, by electing different polymer grades for the inner compartment, fine-tuning of the flexibility of the ring is possible. The thickness of the skin and the vinyl acetate content of the skin influence the release rate of the active ingredient. The thinner the skin and the higher the vinyl acetate content of the skin, the higher the release rate of the active ingredient.

In one embodiment, EVA copolymers having a vinyl acetate content of from 6% to 40% are used. In another embodiment, EVA copolymers having a vinyl acetate content of from 6% to 33% are used. In a further embodiment, EVA copolymers having a vinyl acetate content of from 9% to 33% are used. In yet another embodiment, EVA copolymers having a vinyl acetate content of from 12% to 33% are used. In another embodiment, the skin is made of EVA copolymers having a vinyl acetate content of from 6% to 28%. In yet another embodiment, the skin is made of EVA copolymers having a vinyl acetate content of from 9% to 28%, for example, EVA 9, EVA 15, EVA 18 or EVA 28. It is known in the art that a lower vinyl acetate content of the EVA copolymers results in a higher stiffness of the vaginal ring. Moreover, a larger cross-sectional diameter will also result in a higher stiffness, i.e. less flexibility.

A vaginal ring of the present invention can be manufactured by the known process of extrusion, such as co-extrusion and blend extrusion. To obtain the material for the inner compartment comprising the drug, mirtazapine is mixed with an EVA copolymer. The major step in the mixing process is blend extrusion. Subsequently, the drug/EVA copolymer mixture is co-extruded with the core and skin materials into a three-layered (core comprising) fiber. Alternatively, the drug/EVA copolymer mixture is co-extruded with the skin material into a two-layered fiber (ring without core). After this step, the drug will partly be dissolved in the EVA copolymer. The solubility of the drug in the copolymer is determined by the vinyl acetate content of the EVA copolymer used. Any drug material that is not dissolved will be present as a solid phase in the inner compartment. The solid phase will be in equilibrium with the dissolved phase of the drug, such providing a constant concentration of dissolved active substance close to the rate controlling skin layer. The three-layered or two-layered fiber thus-obtained is cut into pieces of a desired length and each piece is assembled to a ring-shaped device in any suitable manner known to the person skilled in this art. The rings are then packed, for example in a suitable sachet, optionally after being sterilized or disinfected.

A person skilled in the art of extrusion will have no difficulty in finding the optimal processing conditions, such as determining the extrusion temperature, extrusion speed, and air gap, for making a three-layered or two-layered fiber containing drug on the basis of methods and procedures known in the art and the description and examples given in this application. A suitable temperature for blend extrusion of the drug/EVA copolymer mixture lies in the range of from 80° C. to 170° C., e.g. approx. 110° C. Suitable temperatures for co-extrusion of the three-layered or two-layered fiber lie in the range of from 80° C. to 170° C., e.g. from 110° C. to 130° C.

A preferred temperature for extrusion of mirtazapine/EVA copolymer mixtures is below the melting point of the drug, i.e. below approximately 120° C. Melting the drug during extrusion may lead to phenomena like delayed crystallization of the drug.

In this way, vaginal rings with constant release rates of drug, for example releasing in the range of 0.1 to 60 mg/day of mirtazapine, can be manufactured.

The vaginal ring according to the present invention can be manufactured in any practical size. In one embodiment, the ring has an outer diameter of between about 50 and 60 mm and in another embodiment between about 52 and 56 mm. In a further embodiment, the cross-sectional diameter is between about 2.0 and 6.0 mm, in a still further embodiment between about 2.5 and 5.0 mm, in another embodiment between about 3.0 and 4.0 mm, and in yet another embodiment it is about 4.0 mm.

In one embodiment, the amount of drug contained in the inner compartment is from 5 to 80 wt %, in another embodiment from 10 to 70 wt %, in still another embodiment from 30 to 70 wt %, and in a further embodiment from 40-65 wt %.

In another embodiment, the skin is made of EVA copolymers having a vinyl acetate content of from 9% to 28% and the amount of drug contained in the medicated inner compartment is 40-65 wt %.

In one embodiment the drug delivery system according to the invention is a cylindrical fiber, consisting of a cylindrical inner compartment and a skin covering this compartment. In a particular embodiment the cross sectional diameter of such a cylindrical fiber is between about 2.5 and 6 mm, in a specific embodiment between about 3.0 and 5.5 mm, and in another embodiment between about 3.5 and 4.5 mm and in yet another embodiment is 4.0 or 5.0 mm. In one embodiment, the surface of the fiber is more than 800 mm², and in another embodiment more than 1000 mm² and in a further embodiment in the order of 1700-2200 mm². Significantly larger surfaces are possible, provided that the design (physical dimensions) of a drug delivery system intended for vaginal use prevents inconvenience for the subject.

In one embodiment said skin has a thickness in the range of 20 to 200 μm, in another 20 to 100 μm. In a still further embodiment said skin has a thickness in the range of 20 to 70 μm. In a still even further embodiment the copolymer of the inner compartment contains 18 to 33 wt % of vinylacetate. In an even further embodiment the copolymer of the inner compartment contains 28 to 33 wt % of vinylacetate. In an even further embodiment the copolymer of the inner compartment comprises 33 wt % of vinylacetate.

### REFERENCES

A. Kydonieus, Marcel Dekker Inc. New York, 1992. Typical diffusion coefficient for steroids, pp. 66-67.
A. D. Woolfson, R. K. Malcolm, R. J. Gallagher, Journal of Controlled Release 91 (2003) 465-476.

### FIGURE LEGENDS

FIG. 1 shows a cross-sectional presentation of a three-layered drug (core comprising) delivery system in accordance with the present invention.

FIG. 2 shows the in vitro release curves of mirtazapine of three-layered rings with an average release of day 2-14 of approximately 7.5 mg/day. (Batches 7, 10, 13 and 16).

FIG. 3 shows the in vitro release curves of mirtazapine of three-layered rings with an average release of day 2-14 of approximately 15 mg/day. (Batches 6, 11 and 18).

FIG. 4 shows the release rate of a vaginal ring according to the invention compared with a ring, cut into a rod with two open “ring-ends” (Batch 2).

FIG. 5 shows the release rate of a vaginal ring according to the invention with substantially constant release (Batches 11 and 20).

FIG. 6: In vitro release rate (IVR) of 20-70 wt % Mirtazapine containing three-layered rings, wherein the inner compartment comprises 20 (Batch A1), 50 (Batch C1), 60 (Batch D3) and 70 wt % of drug (Batch E1) (341 μm intermediate layer thickness as inner compartment).

FIG. 7: I in vitro release rate (IVR) of mirtazapine containing three-layered rings, wherein the inner compartment comprises 40 (Batch B4), 60 (Batch D4) and 70 wt % of drug (Batch E2) (682 μm intermediate layer thickness as inner compartment)

FIG. 8: In vitro release rate (IVR) of 60 wt % mirtazapine containing three-layered rings, wherein the skin material is EVA 28 (Batch D3) and EVA 15 (Batch D7).

FIG. 9: Side-view of silicone ring and EVA ring having a cross-sectional diameter of 9 and 4 μm respectively.

FIG. 10: View from above of mirtazapine silicone ring and mirtazapine EVA ring having an outer diameter of 54 μm.

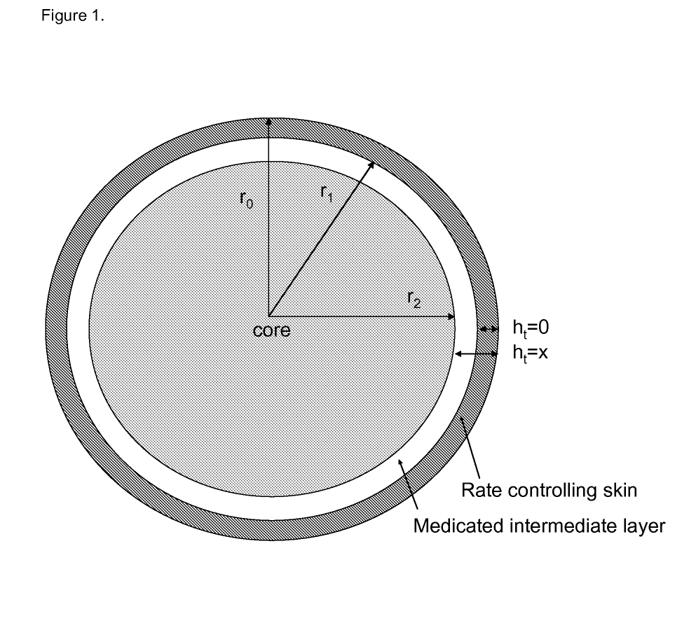

The present invention is illustrated by the following Examples.

### EXAMPLE 1

### Preparation of Three-Layered Vaginal Rings Containing Mirtazapine

Preparation of three-layered vaginal rings consisted of several steps. First of all, an inner compartment granulate containing mirtazapine and EVA 33 copolymer was manufactured in a conventional way by pre-mixing, blend extrusion and lubrication with magnesium stearate. Secondly, a core material of EVA 28 was prepared by lubricating the as-supplied material. Subsequently, the inner compartment granulate, the core granulate and the non-medicated skin material of EVA 28, were co-extruded into a three-layered fiber. The fiber was cut to fibers of a specific length, as described below, after which the fiber ends were welded to a ring.

The inner compartment material was prepared by adding the desired amount (i.e. 60 wt % mirtazapine and 40 wt % EVA 33) of ingredients to a stainless steel drum after which the powder mixture was pre-mixed by rotating the drum on a Rhönrad at 47 rpm for 60 minutes. The powder mixture was subsequently fed to a Berstorff ZE25 co-rotating twin screw extruder and blend extruded at an extrusion temperature of 110° C. Blend extrusion resulted in strands in which mirtazapine was homogeneously dispersed in the EVA copolymer. The strands were subsequently granulated to inner compartment granulate. Prior to co-extrusion, the inner compartment layer granulate was lubricated with 0.1 wt % magnesium stearate and homogenized in a stainless steel drum on a Rhönrad (barrel-hoop principle) with a fixed rotation speed of 47 rpm for 60 minutes.

The core granulate (EVA 28) was also lubricated with 0.1 wt % magnesium stearate and homogenized in stainless steel drum on a Rhönrad (barrel-hoop principle) with a fixed rotation speed of 47 rpm for 60 minutes.

The co-extrusion set-up consisted of a 15 mm skin extruder that processed the skin material, a 18 mm core extruder that processed the core material and an 18 mm inner compartment extruder that processed the inner compartment granulate as delivered by the blend extruder. The melt flows were combined in a spinneret resulting in a three-layered skin—inner compartment—core fiber. The volume flow rate of all three melt flows was controlled by a set of separate spinning pumps. An extrusion temperature of approx 105 to 115° C. and an extrusion rate of 1-2 m/min was used. Extrusion lead to a three-layered fiber with a diameter value of approx. 4 mm, a value of approx. 300 μm for the inner compartment and a skin thickness of approx. 30 μm. The fiber was cooled down to room temperature (RT) in a water bath and wound on a reel. The fiber was cut into 157 mm fibers using a semi-automatic cutter (Metzner) or by hand and subsequently the fibers were welded into a ring at 130° C.

Three-layered rings containing various materials and thicknesses for skin and inner compartment were manufactured (see Table 1). All batches had an EVA 28 core.

TABLE 1
Dimensions of the mirtazapine rings produced comprising a core

| | | | | Inner | | |
|---|---|---|---|---|---|---|
| | | Skin | Inner | compartment | Concentration | Fiber |
| Batch | Skin | thickness | compartment | layer thickness | drug | diameter |
| Fiber | material | (μm) | layer material | (μm) | (wt %) | (mm) |
| 1 | EVA 9 | 200 | EVA 33 | 576 | 40 | 4.1 |
| 2 | EVA 15 | 30 | EVA 33 | 576 | 40 | 4.0 |
| 6 | EVA 28 | 30 | EVA 33 | 576 | 40 | 4.0 |
| 7 | EVA 28 | 200 | EVA 33 | 659 | 40 | 4.1 |
| 10 | EVA 15 | 200 | EVA 33 | 341 | 60 | 4.1 |
| 11 | EVA 28 | 30 | EVA 33 | 341 | 60 | 4.1 |
| 12A | EVA 15 | 30 | EVA 18 | 1018 | 40 | 3.0 |
| 13 | EVA 33 | 30 | EVA 18 | 583 | 40 | 4.0 |
| 14 | EVA 15 | 30 | EVA 18 | 583 | 40 | 3.4 |
| 16 | EVA 15 | 30 | EVA 18 | 344 | 60 | 3.9 |
| 18 | EVA 33 | 30 | EVA 33 | 341 | 60 | 4.1 |
| 20 | EVA 28 | 30 | EVA 33 | 1068 | 60 | 4.0 |

In Vitro Release Rate of Core-Comprising Rings Containing Mirtazapine

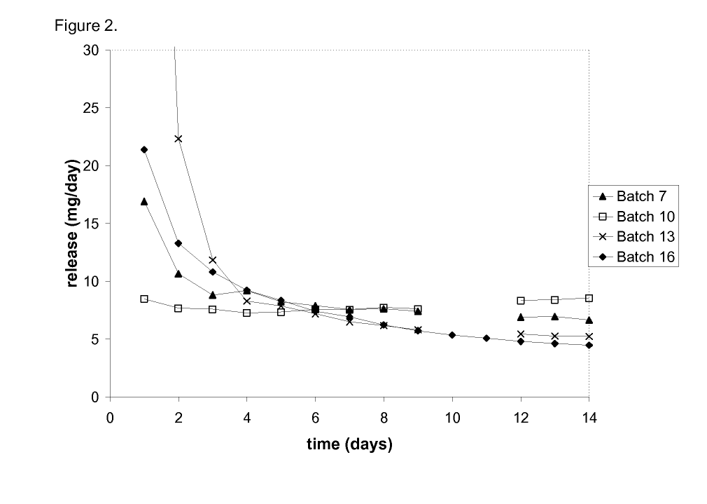

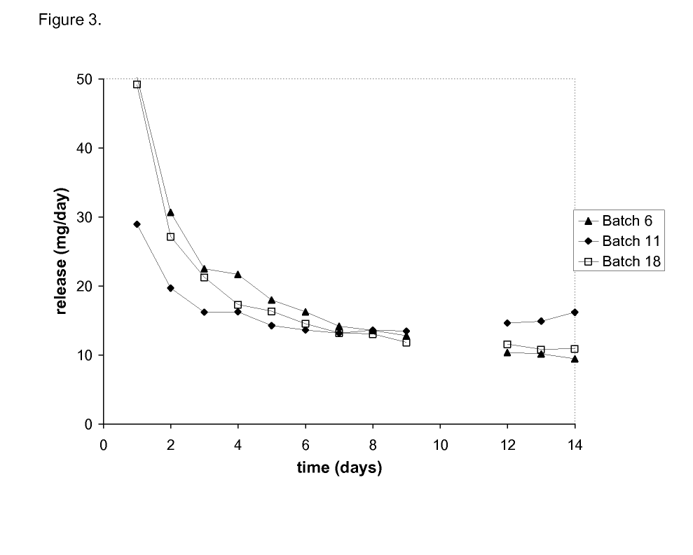

In vitro release rate profiles of the vaginal rings were tested at 37° C. in water for 2 to 4 weeks. The results are presented in Table 2 and of the fibers 7, 10 and 16 in FIG. 2 and of the fibers 6, 11 and 18 in FIG. 3. The results in the figures clearly show that by varying the materials for skin and inner compartment, thickness of skin and inner compartment and concentration of drug (wt %) an average release on days 2-14 of approximately 7.5 mg/day (FIG. 2) and 15 mg/day (FIG. 3) can be achieved. Substantially constant release rates of approximately 25 mg/day are shown in FIG. 5 wherein the release results with batches 10 and 20 are compared.

### Results of Daily Release Rate Measurements

See table 2, next page.

TABLE 2
In vitro release rates of mirtazapine in water

Mirtazapine average release rate
(mg/day)

| Batch | Day 2-14 | Day 2-28 | Day 14 | Day 28 |
|---|---|---|---|---|
| 1 | 1.4 | 1.4 | 1.3 | 1.2 |
| 2 | 12.4 | 9.4 | 8.7 | 5.9 |
| 6 | 16.3 | 11.3 | 9.4 | 5.8 |
| 7 | 8.0 | 6.7 | 6.6 | 5.0 |
| 10 | 7.8 | —* | 8.5 | —* |
| 11 | 15.1 | 13.9 | 16.2 | 6.5 |
| 12A | 8.0 | —* | 5.0 | —* |
| 13 | 8.3 | —* | 5.2 | —* |
| 14 | 6.8 | —* | 4.4 | —* |
| 16 | 7.1 | —* | 4.5 | —* |
| 18 | 15.3 | —* | 10.9 | —* |
| 20 | 23.7 | 24.7 | 25.7 | 23.5 |

—* = not determined

### Conclusions

The in vitro release rate profiles of the vaginal rings as given in FIGS. 2 and 3 show that, after a relatively high rate in the first 2-4 days, the release is prolonged at a constant release rate for periods up to and including 14 days. The initial high rate, that can be considered as a loading dose for fast attaining the desired plasma level in use, is clearly dependent on composition parameters and can be fine-tuned. An average release of day 2-14 of approximately 7.5 mg/day (Table 2: 7, 10 and 16) and 15 mg/day (Table 2: 6, 11 and 18) have been obtained.

A substantially constant release rate of approximately 25 mg/day is shown in FIG. 5.

### EXAMPLE 2

### Test for the Risk of Dose-Dumping

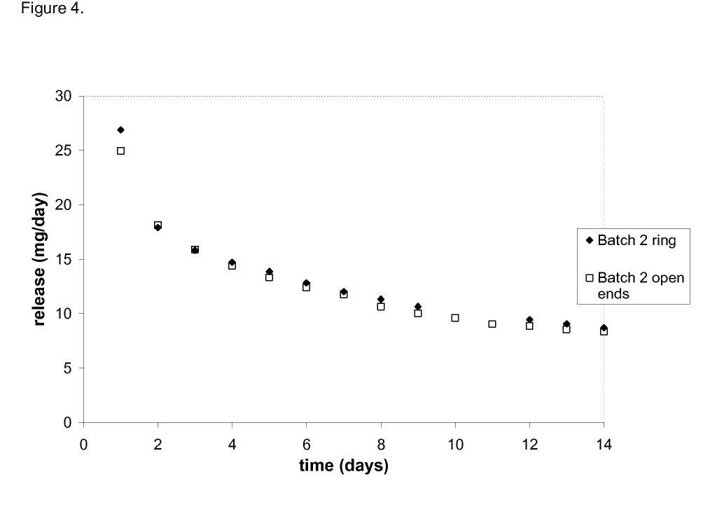

In an in vitro release study in water at 37° C. the mirtazapine release rate of a vaginal ring according to the invention is compared with a ring, cut into a rod with two open “ring-ends”. The in vitro results are depicted in FIG. 4. It is clearly shown that the release rate was not significantly affected, indicating that no dose-dumping occurred. Apparently the design of the device according to the invention inherently protects against dose-dumping problems of high-dose drug delivery systems comprising drugs like mirtazapine.

### EXAMPLE 3

### Effect of Drug Load

Rings were made as specified in the following table 3

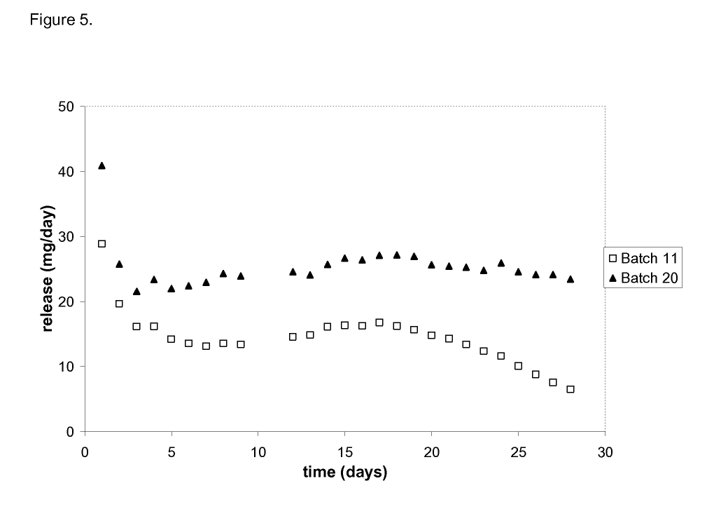

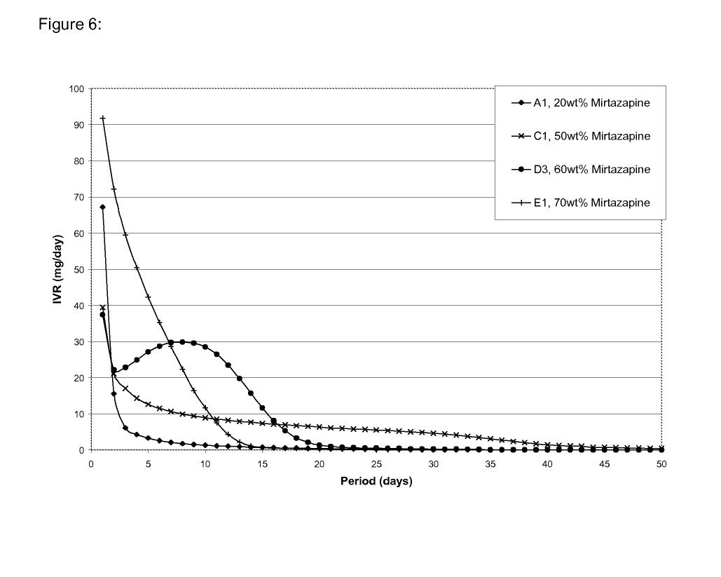

TABLE 3

| | | Inner compartment | | | | | | |
|---|---|---|---|---|---|---|---|---|
| | | | | | Drug | | | Spin |
| Label in | Core | | | | load | Skin layer | | block |
| FIG. 6 | material | wt % | μm | type | (mg) | μm | type | (° C.) |
| A1 | EVA 28 | 20 | 341 | EVA 33 | 121 | 30 | EVA 28 | 105 |
| C1 | EVA 28 | 50 | 341 | EVA 33 | 325 | 30 | EVA 28 | 105 |
| D3 | EVA 28 | 60 | 341 | EVA 33 | 400 | 30 | EVA 28 | 105 |
| E1 | EVA 28 | 70 | 341 | EVA 33 | 479 | 30 | EVA 28 | 105 |

### Conclusion

The effect drug load in the polymer is shown in FIG. 6. The release rate is more constant and substantial over an extended period of days with the rings loaded with 50% and 60%.

### EXAMPLE 4

Rings were made as specified in the following table 4

TABLE 4

| | | | Inner compartment | | | | | | Spin |
|---|---|---|---|---|---|---|---|---|---|
| | | Core | | | | Drug load | Skin layer | | block |
| Batch no | Exp | material | wt % | μm | Type | (mg) | μm | type | (° C.) |
| PD07.32119 | B4 | EVA 28 | 40 | 682 | EVA 33 | 459 | 30 | EVA 28 | 105 |
| PD07.32134 | D4 | EVA 28 | 60 | 682 | EVA 33 | 724 | 30 | EVA 28 | 105 |
| PD07.32139 | E2 | EVA 28 | 70 | 682 | EVA 33 | 868 | 30 | EVA 28 | 105 |

### Conclusion

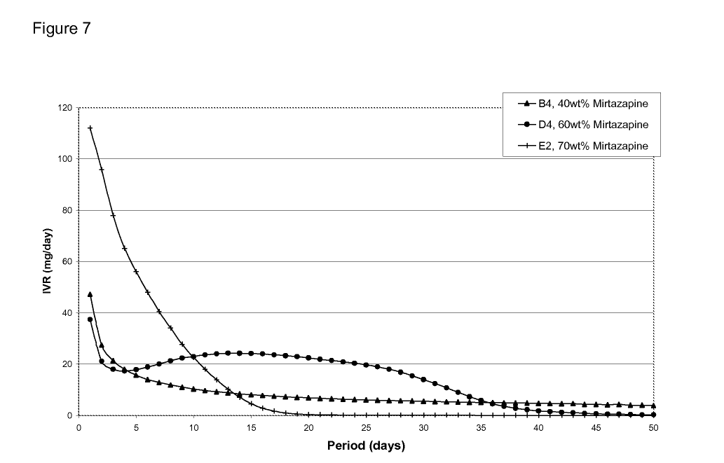

The effect of drug load in the polymer is shown in FIG. 7 with a thicker intermediate layer of 682 μm as inner compartment. The release rate is more constant and substantial over an extended period of days with the rings loaded with 40% and 60%.

### EXAMPLE 5

Rings were made as specified in the following table 5

TABLE 5

| | | | Inner compartment | | | | | | Spin |
|---|---|---|---|---|---|---|---|---|---|
| | | Core | | | | Drug load | Skin layer | | block |
| Batch no | Exp | material | wt % | μm | type | (mg) | μm | type | (° C.) |
| PD07.32133 | D3 | EVA 28 | 60 | 341 | EVA 33 | 400 | 30 | EVA 28 | 105 |
| PD07.32137 | D7 | EVA 28 | 60 | 341 | EVA 33 | 400 | 30 | EVA 15 | 105 |

### Conclusion

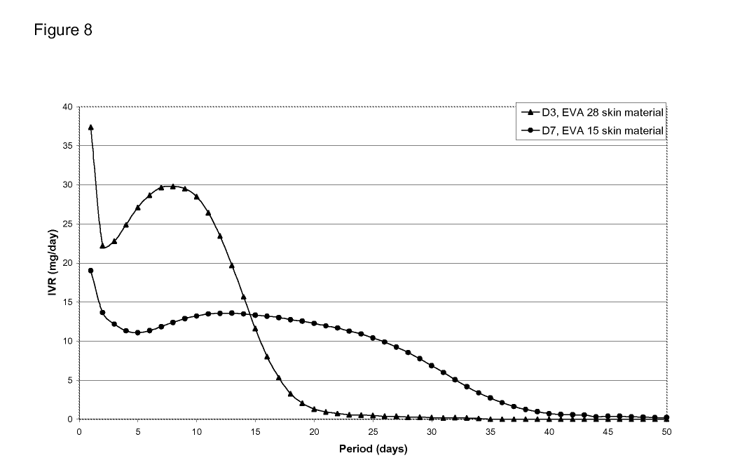

The effect of the use of EVA 15 in comparison to EVA 28 for the skin material is shown in FIG. 8. A more constant and still high release over an extended period is observed for the rings with EVA 15 skin material.

### EXAMPLE 6

### Comparison Mirtazapine Eva Ring with Mirtazapine Silicone Ring

In WO 2005/004837 (Malcolm and Woolfson) a ring for vaginal extended drug release made of polydimethylsiloxane (PDMS) elastomer is described. It was supposed that the release rate could be influenced by making one or more holes or openings extending through the skin to the inner, drug-loaded compartment. By altering the number or size of the openings the release of a salt of an active pharmaceutical agent from the inner compartment could be regulated.

In this example silicone rings are shown based on this technology. In order to restrain gross mobility, either permanently or reversibly, the PDMS was cross-linked by adding a crosslinker: n-propylorthosilicate (NPOS) and a curing catalyst: stannous octoate. Rings having a release rate of mirtazapine of 10-15 mg/day for at least 21 days were made. It was found for mirtazapine that it was not possible to regulate the release by introducing holes in the skin or by making different concentrations of mirtazapine in the inner compartment. Also, the addition of a release-modifying agent had no positive effect on the release. The release rate was regulated by the surface of the ring and in order to increase the surface of the ring for a sufficient release rate a diameter of 9 mm was needed with the silicone ring. As a consequence the silicone ring was far less, i.e. approx. a third to a fourth less flexible than the rings made of EVA polymer.

TABLE 6
Overview of materials used in this example 6.

| | | | Article | Batch | |
|---|---|---|---|---|---|
| No. | Substance | Supplier | number | number | Quantity |
| 1. | MED-6382^{a} | Nusil Technology | — | Lot # 40972 | ~456 g |
| 2. | Stannous Octoate | Nusil Technology | — | Lot # 40972 | ~4 g |
| 3. | Hydroxyethyl | Fluka | 09368 | Lot # 1249544 | ~16 g |
| | cellulose | Biochemika | | | |
| 4. | Mirtazapine | Diosynth | 44.640.300 | L00029018 | ~57 g |
| | | | | L00023933 | |

^{a}The polydimethylsiloxane (MED-6382), obtained from Nusil Technology contained 25 wt % diatomaceous earth, <2 wt % silica, amorphous and <2% terta-n-propyl silicate.

### Equipment

Memert oven, inv. nr: 5231010, temperature range>80° C.
Mould for producing inner compartment
   Material: aluminum
   Outer diameter of ring: 50.5 mm
   Inner diameter of ring: 39.5 mm
   Diameter of cavity of the mould: 5.5 mm
Mould for producing the final (two-layered) ring (see FIGS. 5 and 6)
   Material: aluminum
   Outer diameter of ring: 54 mm
   Inner diameter of ring: 36 mm
   Diameter of cavity of the mould: 9 mm
   8 aluminum pins to obtain holes. Pins are located on the surface of the inner compartment through the outer skin. The pins penetrate the skin up to the inner drug containing compartment
50 mL Braun Omnifix Syringes

In the chosen experimental setup, three parameters were varied: a) The number of holes in the outer layer; b) The concentration of mirtazapine in the inner compartment; c) The addition of pore forming agent to the inner compartment.

In order to investigate the effect of concentration of mirtazapine the concentration was varied between two levels: 10 wt % and 30 wt %. The influence of holes in the skin was examined by varying the number of holes between 8 and 0. Finally, the addition of a release-modifying agent, i.e. a pore forming agent (hydroxylethylcellulose (HEC 30 wt %)) was investigated. All parameter varieties were based on the information given in WO 2005/004837.

The ring composition consists of two layers; a reservoir layer (an inner compartment containing the active material mirtazapine and HEC) and a skin layer containing holes. The two layers were constructed in three stages:

Stage 1] Mixing of mirtazapine with liquid silicone elastomer
Stage 2] Curing of active ingredient containing inner compartment of the ring
Stage 3] Curing of skin material enclosing the inner compartment.

### Ad Stage 1:

An amount of silicone elastomer paste (see table 7) was mixed manually for at least 5 minutes with an amount of mirtazapine. The mixing was complete after 5 minutes, no mirtazapine was visible anymore in the grey silicone paste. The used amounts of materials are depicted in the table 7:

TABLE 7
overview of used compositions of inner compartments^{a}.

| | | Amount | | | |
|---|---|---|---|---|---|
| | Amount of | of | Amount | Conc. | Conc. |
| | Mirtazapine | Silicone | of HEC | Mirtazapine | HEC |
| Batch nr. | [g] | paste [g] | [g] | [wt %] | [wt %] |
| PD07.32225 | 17.65 | 58.81 | — | 30.0 | — |
| PD07.32226 | 4.43 | 16.19 | — | 27.4 | — |
| PD07.32227 | 8.16 | 27.19 | — | 30.0 | — |
| PD07.32228 | 3.30 | 10.06 | — | 32.8 | — |
| PD07.32229 | 4.99 | 16.86 | 5.07 | 29.6 | 30.0 |
| PD07.32230 | 3.28 | 10.89 | 3.25 | 30.1 | 29.9 |
| PD07.32231 | 2.94 | 9.77 | 2.93 | 30.1 | 30.0 |
| PD07.32232 | 4.14 | 13.69 | 4.15 | 30.2 | 30.3 |
| PD07.32233 | 1.87 | 19.91 | — | 9.4 | — |
| PD07.32234 | 1.32 | 13.16 | — | 10.0 | — |
| PD07.32235 | 1.53 | 15.25 | — | 10.1 | — |
| PD07.32236 | 0.81 | 8.79 | — | 9.2 | — |

^{a}The amounts summarized in the table are quantities transferred into the syringe. These are not the amounts which are injected into the two parts of the moulds. These quantities were not determined.

The procedure for mixing HEC in the inner compartment drug reservoir was almost the same as for mixing mirtazapine. HEC was first mixed manually with silicone elastomer for approx. 5 minutes until a homogenous mixture was obtained. Subsequently, the mirtazapine was added and mixed for at least 5 minutes.

### Ad Stage 2.

In order to cure the mixture a total amount of the mirtazapine/(HEC)/silicone mixture, mentioned in table 7, was transferred into a 50 mL syringe after which 1 wt % (with respect to the total mixture) stannous octoate was added as a curing agent. The content of the syringe was mixed in a short time (<2 min) otherwise the mixture cures in the syringe. Subsequently, the grey mixture was injected into the two parts of the mould (upper and lower part). The two parts were tightened together by four overhead screws. The curing process was performed according to three different curing processes:
1] Approx. 5 hours at 80° C. followed by approx. 1 hour cooling to room temperature.
2] Approx. 1 hour at 80° C., followed by cooling overnight in the oven to room temperature.
3] Over a weekend at room temperature

However, during the production of the different batches it was empirically determined that a curing time of approx. 1 hour at 80° C., followed by a cooling period of 1 hour until room temperature was sufficient to produce a ring which was fully cured. In table 8 the amount of stannous octoate and the curing process is summarized.

TABLE 8
overview of curing conditions for preparing the inner compartments^{a}.

| | Amount | Amount of | Conc. | |
|---|---|---|---|---|
| | of | Stan. | Stan. | |
| | mixture | Octoate^{b} | octoate | |
| Batch nr. | [g] | [g] | [wt %] | Curing process |
| PD07.32225 | 16.13 | 0.189 | 1.17 | 3 hr 80° C., cooling |
| | | | | overnight to RT |
| PD07.32226 | 16.70 | 0.162 | 0.97 | 5 hr 80° C., cooling 1 hr |
| | | | | to RT |
| PD07.32227 | 21.56 | 0.215 | 1.00 | 1 hr 80° C., cooling |
| | | | | overnight to RT |
| PD07.32228 | 11.22 | 0.109 | 0.97 | 1 hr 80° C., cooling 1 hr |
| | | | | to RT |
| PD07.32229 | 22.30 | 0.232 | 1.04 | Weekend at RT |
| PD07.32230 | 17.42 | 0.100 | 0.57^{b} | 1 hr 80° C., cooling 1 hr |
| | | | | to RT |
| PD07.32231 | 15.64 | 0.084 | 0.54^{b} | 2 hr 80° C., cooling 1 hr |
| | | | | to RT |
| PD07.32232 | 21.97 | 0.121 | 0.55^{b} | 1 hr 80° C., cooling |
| | | | | overnight to RT |
| PD07.32233 | 19.13 | 0.212 | 1.11 | 1 hr 80° C., cooling |
| | | | | overnight to RT |
| PD07.32234 | 12.99 | 0.095 | 0.73 | 5 hr 80° C., cooling 1 hr |
| | | | | to RT |
| PD07.32235 | 13.52 | 0.162 | 1.20 | 1.5 hr 80° C., cooling 1 hr |
| | | | | to RT |
| PD07.32236 | 8.24 | 0.065 | 0.79 | 1.5 hr 80° C., cooling 1 hr |
| | | | | to RT |

^{a}The amounts summarized in the table are quantities transferred into the syringe. These are not the amounts which are injected into the two parts of the moulds. These quantities were not determined. Instead of calculating the amount of stannous octoate based on the mixture of silicone/mirtazapine the stannous octoate amount was based on the silicone elastomer only. This has no negative influence on the curing time/process.

### Ad Stage 3

The final stage was the enclosure of the inner compartment in the skin. An amount of silicone elastomer paste was quickly mixed (<1 min) with stannous octoate and injected into the two mould parts. Depending on the number of holes in the inner or outer ring, the center piece was placed in the inner compartment and pins were placed in the viscous liquid on the outside of ring. Subsequently the inner compartment with center piece was placed in one of the two parts of the mould. The other mould is placed on the first mould and tightened firmly by tightening the four overhead screws. The curing was performed as described in stage 2. In order to avoid that the pins were pushed outwardly by the curing elastomer, clamps were positioned on the outer pins. In case of a ring were no holes were needed the center piece and the pins in the outer ring were omitted. After curing, flashes and other irregularities were removed.

In table 9 an overview is given of the amount of stannous octoate used for the curing process of the skin. In order to confirm that the holes completely penetrated the skin, the holes were visually examined after IVR on absence of a membrane by two aspects:
presence of interface line on the inner compartment, i.e. during production of the inner compartment the two mould parts were pressed together resulting in a visual interface line between the two parts.

After enclosure of the inner compartment by the skin the line must be visual inside the holes. If not, than it is possible that a thin membrane was formed between the ending of the pin and the inner compartment. Therefore a second visual assessment was performed:
if the line is not present the ring will be cut cross sectional at the position of the holes. By visual assessment the holes will be judged on absence of the membrane.

TABLE 9
overview of curing conditions for preparing the skin^{a}.

| | Amount of | Amount of | Conc. Stan. | |
|---|---|---|---|---|
| | Silicone | Stan. | octoate | |
| Batch nr. | [g] | octoate [g] | [wt %] | Curing process |
| PD07.32225 | 16.15 | 0.180 | 1.11 | Overnight at RT |
| PD07.32226 | 21.84 | 0.216 | 0.99 | 6 hr 80° C., cooling |
| | | | | 1 hr to RT |
| PD07.32227 | 28.12 | 0.288 | 1.02 | 1 hr 80° C., cooling |
| | | | | over weekend to |
| | | | | RT |
| PD07.32228 | 14.34 | 0.135 | 0.94 | Overnight at RT |
| PD07.32229 | 26.64 | 0.254 | 0.95 | 1.5 hr 80° C., |
| | | | | cooling 1 hr to RT |
| PD07.32230 | 11.91 | 0.096 | 0.80 | 1 hr 80° C., cooling |
| | | | | 1 hr to RT |
| PD07.32231 | 19.40 | 0.167 | 0.86 | Overnight at RT |
| PD07.32232 | 19.35 | 0.161 | 0.83 | 1.5 hr 80° C., |
| | | | | cooling 1 hr to RT |
| PD07.32233 | 31.93 | 0.313 | 0.98 | 1 hr 80° C., cooling |
| | | | | 1 hr to RT |
| PD07.32234 | 34.60 | 0.306 | 0.88 | Weekend at RT |
| PD07.32235 | 12.23 | 0.119 | 0.97 | 1.5 hr 80° C., |
| | | | | cooling 1 hr to RT |
| PD07.32236 | 13.17 | 0.103 | 0.78 | 1 hr 80° C., cooling |
| | | | | 1 hr to RT |

^{a}The amounts summarized in the table are quantities transferred into the syringe. These are not the amounts which were injected into the two parts of the moulds. These quantities were not determined.

### In-Vitro Release Determination

After production of the rings, analyses were performed on batches PD07.32225, PD07.3227, PD07.32229, PD07.32231, PD07.3233 and PD07.32235. After mass determination the rings were measured in IVR. The release was determined for 21 days in a buffered solution of sodium acetate trihydrate in water (pH 4.9, pH was adjusted with acetic acid) at 37±0.2° C., sampling interval: 24 hours.

In FIG. 3 the release profiles of the 6 batches are depicted. Also a mirtazapine EVA batch, PD07.32119, is included.

There was no difference in release rate between the 6 silicone ring batches. The batches PD07.32233 and PD07.32235, containing 10 wt % mirtazapine showed a somewhat lower release profile than the other four batches. However, there was no significant difference between the ring with 0 holes (PD07.32235) and the ring with 8 holes (PD07.32233). This was also visible between the four batches with 30 wt % mirtazapine present. The ring batches with 30 wt % mirtazapine (PD07.32225, PD07.32227, PD07.32229 and PD07.32231) show more or less similar release profiles and rates. This demonstrates that the presence of holes and HEC has no significant effect on the release rates. Moreover, these data show that the differences in production (curing times) did not result in changes in release rates.

When comparing the 6 batches silicone rings with an EVA mirtazapine ring (batch PD07.32119, core: EVA 28, inner compartment: EVA 33, 40 wt % mirtazapine, thickness: 682 μm, skin: EVA 28, 30 μm) it is noticeable that the EVA ring has a higher initial burst release and a steeper release profile.

A ring with no release controlling skin was produced (batch PD07.32237) and IVR was measured, for 21 days. This ring should show the maximum achievable release rate. Parallel to this, the dose dumping behavior of two rings was determined.

From batches PD07.32226 (30 wt % mirtazapine) and PD07.32230 (30 wt % mirtazapine, 30 wt % HEC, IVR was 7 days) a piece of approx. 0.5 cm was removed from the ring to create two open fiber ends from which the mirtazapine could be “dumped” to the release medium. The two rings were measured to obtain the possible dose dumping results from a 30 wt % ring and a ring containing 30 wt % HEC.

The IVR results were compared with batch PD07.32225 and PD07.32229.

It was observed that removing a piece of ring to realize dose dumping did not affect the IVR profile. For PD07.32226 a higher burst effect was noticeable, batch PD07.32230 on the other hand was even lower than the ‘regular’ batch PD07.32229. The maximum release curve of PD07.32237 showed an increased release profile with respect to batch PD07.32225 and PD07.32229. The effect of the release controlling membrane is demonstrated by this result. Batch PD07.32237 shows the profile of a typical matrix system while the other batches have the typical characteristics of controlled release systems.

The rings containing HEC showed an increase in mass of approx. 0.3 g. This increase was most likely caused by the fact that the silicone skin is somewhat permeable for water in combination with the presence of HEC in the inner compartment. Furthermore, HEC is hygroscopic, hence the increase of mass after IVR. The total amount of absorbed water was approx. 0.6 g. The mass loss of the other rings could be explained by the released amount of mirtazapine.

### Flexibility Test (Pressure Test)

Flexibility tests were performed on batches PD07.32228, PD07.32232, PD07.32234 and PD07.32236 and two representative batches of EVA mirtazapine rings, PD07.32119 and PD07.32137. The flexibility of the ring was determined by means of a compression test. A ring sample is positioned in its relaxed state (approx. 54 mm distance) between two holders. The two holders are moved with a speed of 50 mm/min to each other until the holders have a distance of approx. 21 mm. The forces to compress the ring are recorded at different compressions.

The following table 10 gives the results obtained for 4 different silicone rings.

TABLE 10
Pressure test results of EVA ring (batch PD07.32137 (n = 4) and
PD07.32119 (n = 4)) and 4 different silicone mirtazapine rings
(PD07.32228-PD07.32236).

| | | | Load at 30 mm |
|---|---|---|---|
| Batch | Load at 10 mm (N) | Load at 20 mm (N) | (N) |
| PD07.32137 | 1.3805 | 2.2728 | 4.5155 |
| PD07.32137 | 1.2017 | 1.9539 | 4.2025 |
| PD07.32137 | 1.6896 | 2.6437 | 4.7150 |
| PD07.32137 | 1.4804 | 2.3820 | 4.4535 |
| PD07.32119 | 1.1388 | 1.9476 | 3.9156 |
| PD07.32119 | 1.4795 | 2.2962 | 4.2307 |
| PD07.32119 | 1.6288 | 2.7136 | 5.0292 |
| PD07.32119 | 1.0540 | 1.7557 | 3.5217 |
| PD07.32128 | 3.2920 | 6.7030 | 16.928 |
| PD07.32132 | 4.1029 | 8.4870 | 20.622 |
| PD07.32134 | 2.7500 | 5.6155 | 13.352 |
| PD07.32136 | 2.8738 | 5.9380 | 14.828 |

According to the flexibility test the silicone ring is significant stiffer than the EVA ring. The forces to compress the silicon ring are approx. 3-4 times higher as compared to the EVA rings.

### Dimensions/Appearance of the Rings

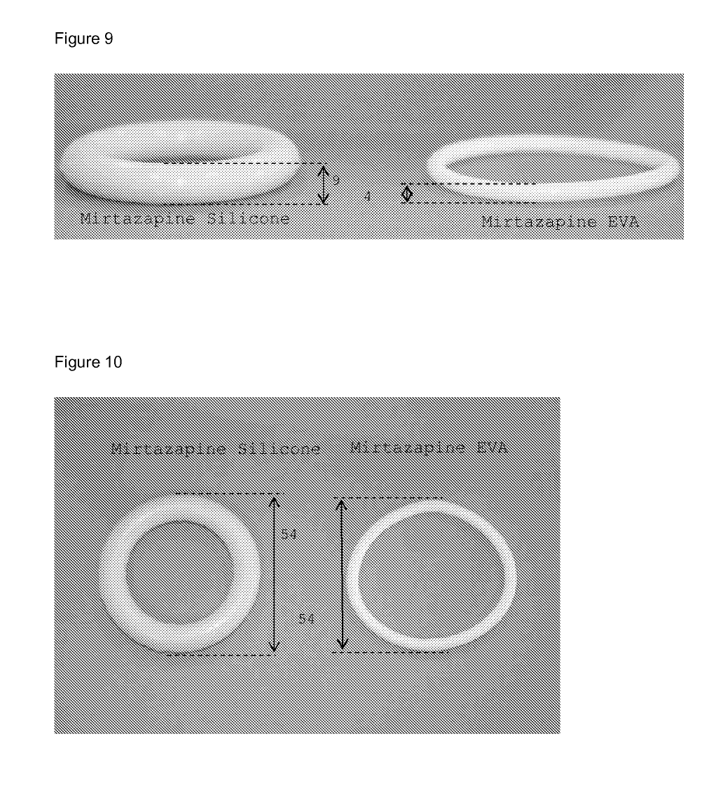

In FIGS. 9 and 10, the typical examples of a silicone and an EVA ring are given. The outer diameter is identical, but the fiber thickness of the silicone ring is substantially higher (9 mm) as compared to an EVA ring (4 mm).

### CONCLUSION

Although there is not much difference between the silicone and EVA rings for a release of approx. 10-15 mg/day for at least 21 days, the silicone mirtazapine rings provide hardly any possibilities to regulate the release in contrast to the EVA ring, in which the release can be controlled by adjusting the thickness and drug loading of the inner compartment and/or thickness of the skin. Therefore the EVA mirtazapine ring has a considerable advantage in use as a intravaginal drug delivery device over the silicone intravaginal ring. Moreover the silicone ring for sufficient mirtazapine release is heavier and much stiffer than the EVA ring. The forces to compress the silicone ring are approximately 3-4 times higher as compared to the EVA rings.

1-9. (canceled)

10. An extended release formulation comprising solid mirtazapine, wherein the formulation is in the form of a vaginal device comprising:
(a) an inner compartment made of a thermoplastic polymer, wherein the polymer contains solid mirtazapine; and
(b) a skin.

11. The formulation of claim 1, wherein the polymer contains 40 to 60 wt % mirtazapine.

12. The formulation of claim 1, wherein the skin is substantially continuous.

13. The formulation of claim 1, wherein the device is a ring having a cross-sectional diameter between about 2.0 and 6.0 mm.

14. The formulation of claim 1, wherein the inner compartment comprises a core, which does not contain solid mirtazapine.

15. The formulation of claim 1, wherein the thermoplastic polymer of the inner compartment is ethylene-vinyl acetate copolymer.

16. The formulation of claim 1, wherein the skin is made of ethylene-vinyl acetate copolymer.

17. The formulation of claim 15, wherein the ethylene-vinyl acetate copolymer of the inner compartment has a vinyl acetate content of 6 to 40%.

18. The formulation of claim 16, wherein the ethylene-vinyl acetate copolymer of the skin has a vinyl acetate content of 6 to 40%.

19. The formulation of claim 18, wherein the ethylene-vinyl acetate copolymer of the skin has a vinyl acetate content of 9 to 18%.

20. A method for treating depression in a patient in need of treatment, the method comprising administering to the patient an extended release formulation of claim 1.

21. The method of claim 20, wherein the depression is major depression.

22. A method for treating hot flush in a patient in need of treatment, the method comprising administering to the patient an extended release formulation of claim 1.

23. A method for treating sleep disorders in a patient in need of treatment, the method comprising administering to the patient an extended release formulation of claim 1.

24. A method for treating menopausal complaints in a patient in need of treatment, the method comprising administering to the patient an extended release formulation of claim 1.
